(19) United States
(12) Patent Application Publication
(10) Pub. No.: US 2013/0337043 A1
(43) Pub. Date: Dec. 19, 2013

(54) Composition Used for Thermotherapy and Producing Method Thereof and Method to Treat Cancer

(71) Applicant: Uropro Biotech Co., Ltd., Taipei City (TW)

(72) Inventors: Kwan-Hwa CHI, Taipei City (TW); Kuang-Wen LIAO, Hsinchu City (TW); Yu-Shan WANG, Taipei City (TW)

(21) Appl. No.: 13/907,504

(22) Filed: May 31, 2013

(30) Foreign Application Priority Data
Jun. 18, 2012 (TW) 101121795

(51) Int. Cl. A61K 41/00; A61N 2/00

(52) U.S. Cl. CPC A61K 41/0052; A61N 2/004
USPC 424/450; 600/12

(57) ABSTRACT

A composition used for thermotherapy includes a carrier structure and a plurality of metal particles. The carrier structure includes a lipid layer, a positive charged polymer and a surface active polymer. The positive charged polymer and the surface active polymer are dispersed on the lipid layer by non-covalent bonding. The metal particles are encapsulated in the carrier structure. A producing method of the composition used for thermotherapy and a method for using the composition in cancer treatment are also disclosed.

### CROSS REFERENCE TO RELATED APPLICATIONS

This Non-provisional application claims priority under 35 U.S.C. §119(a) on Patent Application No(s). 101121795 filed in Taiwan, Republic of China on Jun. 18, 2012, the entire contents of which are hereby incorporated by reference.

### BACKGROUND OF THE INVENTION

1. Field of Invention

The present invention relates to a composition used for thermotherapy, a producing method thereof, and a method thereof to treat cancer.

2. Related Art

Cancer, known medically as a malignant neoplasm, is a broad group of various diseases, all involving unregulated cell growth, and it is extremely hard to be prevented and healed. In cancer, cells divide and grow uncontrollably, forming malignant tumors, and invade nearby parts of the body. The cancer may also spread to more distant parts of the body through the lymphatic system or bloodstream, thereby seriously interrupting the normal physiological functions.

Cancer is usually treated with surgery, chemotherapy, and radiation therapy. Surgery is the primary method of treatment of most isolated solid cancers and may play a role in palliation and prolongation of survival. In localized cancer surgery typically attempts to remove the entire mass along with, in certain cases, the lymph nodes in the area. Chemotherapy is to administrate some medicines, which can affect the division, growth or survival of cancer cells, to inhibit or destroy the cancer cells. Radiation therapy involves the use of ionizing radiation in an attempt to either cure or improve the symptoms of cancer by non-invasive and non-pharmacological treatment.

However, the above-mentioned methods all have limitations. Regarding to surgery, it is suitable for short-term curative and is usually suggested for the initial stage of cancer. Unfortunately, when the cancer develops into the second stage or later, the healing rate by surgery is dramatically decreased.

In general, the medication used in chemotherapy does not have specificity. However, although the chemotherapy medication can kill the tumor cells, it is also toxicity to other normal tissues in the body and causes some symptoms such as hair loss, skin eruption, oral mucositis, diarrhea, constipation, paresthesia, taste loss, and bone marrow suppression. In particularly, the toxicity to digestive track usually causes different levels of nausea and vomit. Recently, the targeted therapy has been developed for attacking the cancer cells in high specificity, thereby sufficiently improving the side effects of chemotherapy medication. However, the targeted therapy is very expensive and the tissues may gradually generate drug resistance, so it is not a so perfect treatment.

The radiation therapy is to either cure or improve the symptoms of cancer. However, during the treatment, the ionizing radiation may also affect the function of normal cells and even more kill a huge amount of normal cells, so it should be very careful in using the radiation therapy.

Currently, it is desired to find a proper therapy.

The past researches indicate that cancer cells or tumor cells can be effectively killed by high temperature, so that the thermotherapy has been disclosed for treating cancer and tumor cells. Thermotherapy can provide heat to tumors by directly contacting with the heat source or by irradiating with infrared ray. However, the conventional thermotherapy is applied to the entire human body, so it is lack of specificity. Accordingly, the conventional thermotherapy can not effectively kill the cancer or tumor cells, and moreover, speed the blood circulation to cause bleeding as the patient has wounds inside, or cause some relative symptoms as the patient has heart disease such as cardiopathy. Since the thermotherapy has superior effects on chemotherapy and radiation therapy, it is expected to have broader applications if the above-mentioned drawbacks are solved. In brief, thermotherapy needs some improvements, especially in issue of heating at the specific position. These improvements are very critical to the future cancer therapy.

Therefore, it is an important subject to provide a thermotherapy technology that can effectively improve the local heating effect so as to decrease the damage to the normal cells, and can directly kill or assist to kill cancer cells so as to broaden the thermotherapy applications.

### SUMMARY OF THE INVENTION

In view of the foregoing, an objective of the present invention is to provide a composition containing metal particles and a producing method thereof as well as a method for using the composition in cancer treatment. The special configuration and structure of the composition can provide an excellent carrier function so that the local heating effect can be easily achieved. Besides, the composition is easily produced and has low cost and broader applications.

Since the composition can be easily connected with the targeted subjects, a preferred objective of the present invention is to further enhance the thermotherapy specificity of the composition, thereby decreasing the side effects and directly or indirectly improve the cancer treatment.

To achieve the above objectives, the present invention discloses a composition used for thermotherapy, comprising a carrier structure and a plurality of metal particles. The carrier structure comprises a lipid layer, a positive charged polymer and a surface active polymer. The positive charged polymer and the surface active polymer are dispersed on the lipid layer by non-covalent bonding. The metal particles are encapsulated in the carrier structure.

In one embodiment of the present invention, the carrier structure comprises liposome.

In one embodiment of the present invention, the carrier structure comprises temperature-sensitive liposome.

In one embodiment of the present invention, the ratio of the lipid layer, the positive charged polymer and the surface active polymer is between 3:1:1 and 60:1:1.

In one embodiment of the present invention, the lipid layer is a neutral lipid layer comprising DLPC, DOPC, DMPC, DPPC, DSPC, DOPC, DMPE, DPPE, DOPE, DMPA, DPPA, DOPA, DMPG, DPPG, DOPG, DMPS, DPPS, or DOPS.

In one embodiment of the present invention, the positive charged polymer comprises polyamine, polyethylenimine (PEI), polyvinylpyrrolidone, or polyacetic acid.

In one embodiment of the present invention, the surface active polymer comprises crosslinked type polyacrylate salt, saponin, or Polyethylene glycol (PEG).

In one embodiment of the present invention, the carrier structure further comprises a targeted subject dispersed on the lipid layer by non-covalent bonding.

In one embodiment of the present invention, the targeted subject comprises antibody, peptide, nucleic acid, or cytokine.

In one embodiment of the present invention, the composition is applied in magnetic resonance thermotherapy, wherein the metal particles are magnetic metal particles.

In one embodiment of the present invention, the metal particles are made of iron, gold, or copper.

In one embodiment of the present invention, the metal particles are subjected to a magnetic force or an electromagnetic wave to generate heat.

The present invention also discloses a producing method of a composition used for thermotherapy, comprising the steps of: forming a lipid layer; adding a solution containing a positive charged polymer and a solution containing a surface active polymer to contact with the lipid layer; shaking the lipid layer with the solutions to form a carrier structure; and encapsulating a plurality of metal particles in the carrier structure.

In one embodiment of the present invention, the producing method of a composition used for thermotherapy further comprises the step of: adding a solution containing a targeted subject to contact with the carrier structure.

In addition, the present invention also discloses a method for using a composition used for thermotherapy to treat cancer. The composition comprises a carrier structure comprising a lipid layer, a positive charged polymer dispersed on the lipid layer by non-covalent bonding and a surface active polymer dispersed on the lipid layer by non-covalent bonding, and a plurality of metal particles encapsulated in the carrier structure.

In one embodiment of the present invention, the carrier structure comprises temperature-sensitive liposome.

In one embodiment of the present invention, the ratio of the lipid layer, the positive charged polymer and the surface active polymer is between 3:1:1 and 60:1:1.

In one embodiment of the present invention, the composition is applied in magnetic resonance thermotherapy, wherein the metal particles are magnetic metal particles.

In one embodiment of the present invention, the metal particles are subjected to a magnetic force or an electromagnetic wave to generate heat.

As mentioned above, the composition used for thermotherapy of the invention includes a carrier structure with a special configuration, so that the carrier structure can effectively encapsulate the metal particles during the preparation process. Based on the property of the lipid layer that can fuse with and pass through the cell membrane, the metal particles can be effectively released to a specific position, such as a specific tumor cell. Since the metal particles can absorb the additionally provided energy and generate vibration to produce high temperature for killing the tumor cells. In addition, the metal particles do not enter other normal cells, so the normal cells will not be affected by the high temperature.

Moreover, the carrier structure of the composition of the invention is suitable for connecting with targeted substances for improving the guiding function. This improved guiding function allows sending the composition to a specific tumor cell, thereby enhancing the thermotherapy specificity and decreasing the side effects. The producing method of the composition of the invention is simple and low cost. It can prepare the desired composition in a short time and increase the applications.

The invention discloses a composition, a producing method of the composition and a method for using the composition to treat cancer, which can improve the conventional cancer therapy technologies, such as the side effects of chemotherapy and radiation therapy, and avoid the complex limitations as providing thermotherapy to the entire body. The preparation of the composition of the invention is easy, so that the cost for cancer treatment can be decreased. In addition, the present invention has extremely high cost effectiveness so as to prevent the drawbacks of the conventional physical therapy, which needs a lot of manpower and time, and has poor effect.

### BRIEF DESCRIPTION OF THE DRAWINGS

The invention will become more fully understood from the detailed description and accompanying drawings, which are given for illustration only, and thus are not limitative of the present invention, and wherein:

FIG. 1 is a schematic diagram showing a composition used for thermotherapy according to an embodiment of the present invention; and

FIG. 2 is a flowchart of the producing method of the composition used for thermotherapy according to the embodiment of the present invention.

### DETAILED DESCRIPTION OF THE INVENTION

The present invention will be apparent from the following detailed description, which proceeds with reference to the accompanying drawings, wherein the same references relate to the same elements.

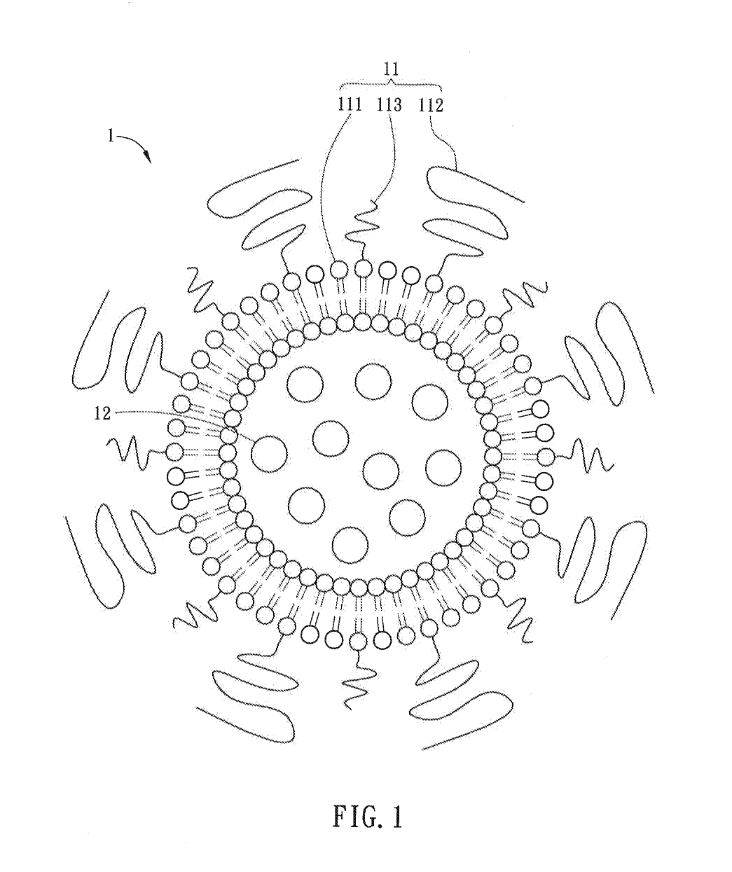

FIG. 1 is a schematic diagram showing a composition used for thermotherapy according to an embodiment of the present invention. Referring to FIG. 1, the composition 1 includes a carrier structure 11 and a plurality of metal particles 12. The carrier structure 11 includes a lipid layer 111, a positive charged polymer 112, and a surface active polymer 113. The positive charged polymer 112 and the surface active polymer 113 are dispersed on the lipid layer 111 by non-covalent bonding. The metal particles 12 are encapsulated in the carrier structure 11. The carrier structure 11 will be described firstly hereinafter.

In one embodiment, the lipid layer 111 is a dual-layer structure mainly composed of neutral lipid, which is for example but not limited to DLPC (dilinoleoylphosphosphatidylcholine), DOPC (dioleoyl-phosphatidylcholine), DMPC (dimyristoyl phosphatidyl-choline), DPPC (dipalmitoylphosphatidylcholine) DSPC (disaturated phosphatidylcholine), DMIPE (dimyristoylphosphatidylethanolamine) DPPE (1,2-Bis-(diphenylphosphino)ethane) DOPE (dioleoylphosphatidyl-ethanolamine), DMPA (dimethylolpropionic acid), DPPA (diphenylphosphoryl azide), DOPA (dioleoylphosphatidic acid), DMPG (dimyristoylphosphatidylglycerol), DPPG (dipalmitoyl phosphatidylglycerol), DOPG (dioleoyl phosphatidyl glycerol), DMPS (dimyristoylphosphatidyl-serine), DPPS (dipalmitoyl phosphatidylserine), or DOPS (dioleoylphosphatidylserine). In a preferred aspect, the lipid layer 111 is substantially composed of DLPC and DOPC. In another embodiment, the fluorescent dye is added during the preparation process, so that the lipid layer has fluorescent characteristic, which is benefit in tracking the composition.

When the composition 1 is injected and located close to an individual cell, the hydrophobic and hydrophilic ends of the lipid layer 111 allows the composition 1 to fuse with cell membrane and than enter the cell. The term “individual cell” is preferably a cell strain cultivated in vitro or in vivo (e.g. in mammals such as mouse, human, cow, sheep, pig, monkey, dog, cat, and the likes, and preferably in human). For example, the cell strain is a mammal cell strain.

Since the lipid layer 111 has a dual-layer structure, the positive charged polymer 112 and the surface active polymer 113 are dispersed on the inner and outer surfaces of the lipid layer 111 by non-covalent bonding. The non-covalent bond is formed faster than the normal covalent bond and has lower energy threshold, so it is benefit in increasing the production performance.

In this embodiment, the positive charged polymer 112 is a long-chain polymer carrying positive charges, which may include polyamine, PEI (polyethylenimine), polyvinylpyrrolidone, or polyacetic acid, and the surface active polymer 113 may include crosslinked type polyacrylate salt, saponin, or PEG (Polyethylene glycol). However, the invention is not limited to this. Preferably, the positive charged polymer 112 is PEI (polyethylenimine), and the surface active polymer 113 is PEG (Polyethylene glycol).

The positive charged polymer 112 and the surface active polymer 113 can be dispersed on the surfaces of the lipid layer 111 by any desired distributions, or embedded in the structure of the lipid layer II with their long-chain part, or their combination. This invention is not limited and any configuration can be used based on the selected substances. Preferably, some of the long-chain parts of the positive charged polymer 112 and the surface active polymer 113 are embedded in the inner and outer surfaces of the lipid layer 111, so that the positive charged polymer 112 and the surface active polymer 113 are dispersed on and stably combined with the surfaces of the lipid layer 111. The term “non-covalent bonding” includes all reaction force formed by hydrophobicity, hydrophilicity, hydrogen bonding, or van der Waals' force.

The ratio of the lipid layer 111, the positive charged polymer 112, and the surface active polymer 113 is between 3:1:1 and 60:1:1. In this embodiment, the ratio of the lipid layer 111, the positive charged polymer 112, and the surface active polymer 113 is between 10:3:3 and 30:1:1, and is preferably 3:1:1.

With a specific ratio, the lipid layer 111, the positive charged polymer 112, and the surface active polymer 113 can form a closed carrier structure 11, and the shape of the carrier structure 11 is, for example but not limited to, a sphere, an oval, or other irregular 3D shapes. The carrier structure 11 has a chamber therein for encapsulating a plurality of metal particles 12. In this embodiment, the carrier structure 11 is also called a liposome.

The material of the metal particles 12 is preferably, but not limited to, iron, gold or copper; however, any material that can absorb the energy of magnetic force or electromagnetic wave and release heat is applicable. In this embodiment, the metal particles 12 are neutral magnetic metal particles, which are made of Fe₃O₄ or Fe₂O₃. After absorbing the energy of magnetic force or electromagnetic wave, the temperature of the metal particles 12 is increased to between 42° C. and 48° C. or higher, and preferably between 42° C. and 43° C. Besides, the metal particles 12 may further include zinc, cobalt, or nickel for enhancing the magnetic control property.

The metal particles 12 can be mixed with the lipid layer 111, the positive charged polymer 112 and the surface active polymer, and then encapsulated within the carrier structure 11 during the producing process. Otherwise, the metal particles 12 are mixed with other substances such as gel or aqueous solution to form a preferred size, and then encapsulated within the chamber of the carrier structure 11. To be noted, the size of the metal particles is between 2 and 400 ram, and preferably less than 100 mm, and more preferably between 10 mm and 30 mm. Of course, the shape of the metal particles is not limited and can be, for example, a sphere, a tube, a shell, a bulb or the likes.

The lipid layer 111, the positive charged polymer 112, and the surface active polymer 113 can easily form the carrier structure 11. Accordingly, when the carrier structure 11 and the metal particles 12 are mixed to produce the composition 1 of the invention, the large-sized metal particles 12 can be easily encapsulated so as to improve the thermotherapy effect. Besides, since the lipid layer 111, the positive charged polymer 112, the surface active polymer 113, and the metal particles 12 can form the composition 1 by vibration mixing, the manufacturing procedure is simplified, and the preparation time is reduced.

The carrier structure 11 of the invention is a modified liposome with temperature-sensitive property, which is also called as a temperature-sensitive liposome. When the temperature is increased, the carrier structure 11 will be decomposed. Thus, as the composition 1 is applied to the thermotherapy, it is possible to apply heat in larger range or entire body for breaking the carrier structure 11 so as to release the metal particles 12, and then to heat a specific position for vibrating the metal particles 12 so as to generate a higher temperature. This application can provide both accuracy and therapy functions.

In addition, the composition is suitable for the application in vivo or in vitro. In another embodiment, a targeted substance can be applied for guiding the composition to the desired target. The targeted substances can be absorbed on the lipid layer of the carrier structure by non-covalent bonding based on the micro environment on the surface of the lipid layer composed of the lipid layer, the positive charged polymer, and the surface active polymer. Of course, the targeted substances can also be combined with the positive charged polymer and the surface active polymer by charge attraction or by covalent or non-covalent bonding. The targeted substance of the invention comprises antibody, peptide or nucleic acid with specific sequence, or cytokine, which can identify some specific targets in an individual cell such as antigen, antibody, peptide or nucleic acid. In this embodiment, the targeted substance is an antibody or a ligand that can identify the tumor cell in an individual cell with high specificity.

Of course, it is possible to connect some substances on the surface of the carrier structure to mark or track the position of the composition. These substances may comprise, for example but not limited to, a color substance or an irradiation substance.

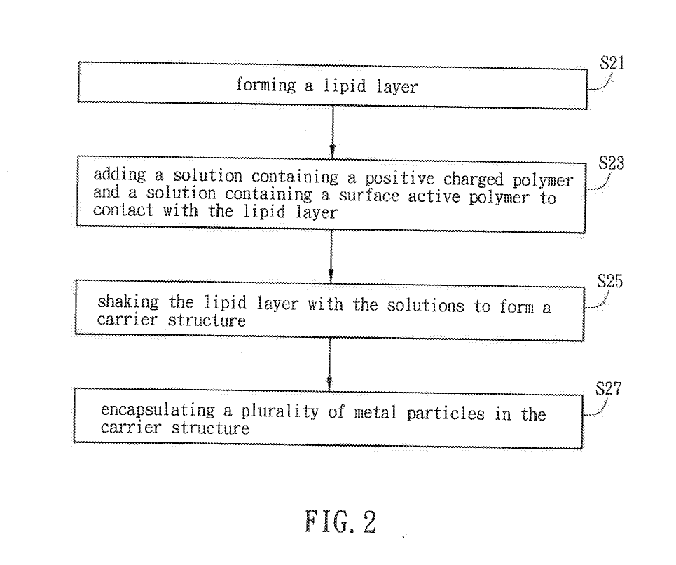

Moreover, the present invention also discloses a producing method of the above-mentioned composition. FIG. 2 is a flowchart of the producing method of the composition used for thermotherapy according to the embodiment of the present invention. As shown in FIG. 2, the producing method of the composition used for thermotherapy includes the following steps of: forming a lipid layer (S21); adding a solution containing a positive charged polymer and a solution containing a surface active polymer to contact with the lipid layer (S23); shaking the lipid layer with the solutions to form a carrier structure (S25); and encapsulating a plurality of metal particles in the carrier structure (S27).

In the step S21, a lipid solution, which contains any of the above-mentioned neutral lipid layers, is added into a pear-shaped flask. After rotary evaporation to remove the solvent, multiple neutral lipid layers are remained at the bottom of the flask. Afterwards, the solutions containing the positive charged polymer and the surface active polymer are added into the same flask so that the solutions are in contact with the lipid layer (step S23). Similarly, the positive charged polymer and the surface active polymer can be any of the above-mentioned compounds. To be noted, the ratio of the lipid layer, the positive charged polymer and the surface active polymer is between 3:3:3 and 30:1:1, preferably between 10:3:3 and 30:1:1, and more preferably 3:1:1.

In the step S25, the lipid layer, the solution containing a positive charged polymer and the solution containing a surface active polymer are shaken by manual or machine to evenly distribute the positive charged polymer and the surface active polymer on the lipid layer, so that they can form a closed sphere carrier structure with a central hollow chamber by hydration. Besides, the positive charged polymer and the surface active polymer have hydrophobic and hydrophilic properties, so that they can be dispersed on the lipid layer by non-covalent bonding. After forming the carrier structure, in another embodiment, the producing method of the composition used for thermotherapy further comprises a step of: pushing the carrier structure through a pore membrane to obtain a plurality of carrier structure in equivalent size. This step can increase the unity of the composition and thus facilitate the following application thereof. In this embodiment, the size of the carrier structure is between 2 and 400 nm, and preferably smaller than 100 nm.

In the step S27, while the carrier structure is formed, the carrier structure simultaneously encapsulates a plurality of metal particles therein. Similarly, the feature and description of metal particles can be referred to the above illustrations. In detailed, the step S27 can be performed by the conventional shaking and vibration approaches, or the complex supercritical fluid (carbon dioxide) technology. The supercritical fluid (carbon dioxide) technology is to add liquid carbon dioxide as the lipid layer, the solution containing the positive charged polymer, and the solution containing the surface active polymer, and then to heat and press the container so as to form the supercritical fluid (carbon dioxide). Afterwards, the carbon dioxide exhausted from the container makes the carrier structure to encapsulate the metal particles. In this case, the surfaces of the metal particles will connect organic compounds in advance and then encapsulated by the carrier structure.

The details of the above steps are well-known to those skilled in the art, so they are not described hereinbelow. The metal particles can be customized metal particles or selected from the commercialized products, and this invention is not limited. Preferably, the metal particles are magnetic metal particles made of Fe₃O₄.

In the step of mixing the metal particles, the positive charged polymer, and the surface active polymer, the ratio of the lipid layer, the positive charged polymer, the surface active polymer, and the metal particles is between 10:3:3:1 and 30:1:1:0.5. Otherwise, it is also possible to prepare the carrier structure in advance and then mix the metal particles with the carrier structure, so that the metal particles are directly encapsulated within the chamber inside the carrier structure.

Moreover, in order to connect the targeted substances on the surface of the lipid layer of the carrier structure to improve the positioning and direction properties of the composition, the producing method of the invention further includes the step of: adding a solution containing a targeted substance to contact with the carrier structure. The preparation of the targeted substance is also well-known to those skilled in the art, so the detailed description thereof will be omitted. The solution containing the targeted substance can be added as the positive charged polymer and the surface active polymer are mixed, or after the carrier structure has been prepared.

The present invention also provides a method for using the composition used for thermotherapy in cancer treatment for curing, eliminating, or inhibiting the in vivo tumor tissues or cells. The tumors to be treated by the composition include benign tumors or malignant neoplasm of any phases, which may exist in breast, stomach, intestine, thyroid gland, uterus, liver, pancreas or the likes.

In practice, the above-mentioned composition (0.05-10 mg/mL) is injected into the individual body for treating a tumor tissue (in vivo, 0.5-10 cm). Since the composition comprises the targeted substances, it can be controlled to stay at a specific tumor tissue. The injection may include intravenous injection, subcutaneous injection, intraperitoneal injection, intramuscular injection, or direct injection to the tumor tissue.

After injecting the composition for a while, the composition has been transmitted to the specific position and passed through the cell membrane of the tumor cell. The large-range or entire body heating is conducted to decompose the carrier structure so as to release the metal particles into the tumor cells. Then, a proper magnetic force or electromagnetic wave is applied to the tumor for providing energy to the metal particles, and the metal particles absorb the energy to vibrate so as to be heated to the temperature between 42° C. and 45° C.

In the above embodiment, the energy is provide by an infrared ray generator, wherein the provided frequency is between 20 and 300 kHz, and the magnetic field is between 5 and 15 kA/m.

Regarding to a tumor cell strain, the composition solution (0.05-10 mg/mL) is applied to treat 1×10⁵-1×10⁷ cell strains, and the same energy is provided to increase the temperature of the composition to 42-45° C. Finally, the size of the tumor tissue or the bioactivity of the tumor cell strain is measured to proof the effectiveness of the composition of the invention in cancer treatment. The methods for measuring the size of the tumor tissue or the bioactivity of the tumor cell strain are well-known by those skilled in the art, so they are omitted hereinafter. Based on the experimental results, the composition of the present invention can effectively decrease the size of the tumor tissue or the survival number of the tumor cell strain. Furthermore, the composition used for thermotherapy or the composition prepared by the producing method of the invention has therapeutic effects in the cancer treatment.

In summary, the composition used for thermotherapy of the invention includes a carrier structure with a special configuration, so that the carrier structure can effectively encapsulate the metal particles during the preparation process. Based on the property of the lipid layer that can fuse with and pass through the cell membrane, the metal particles can be effectively released to a specific position, such as a specific tumor cell. Since the metal particles can absorb the additionally provided energy and generate vibration to produce high temperature for killing the tumor cells. In addition, the metal particles do not enter other normal cells, so the normal cells will not be affected by the high temperature.

Moreover, the carrier structure of the composition of the invention is suitable for connecting with targeted substances for improving the guiding function. This improved guiding function allows to send the composition to a specific tumor cell, thereby enhancing the thermotherapy specificity and decreasing the side effects. The producing method of the composition of the invention is simple and low cost. It can prepare the desired composition in a short time and increase the applications.

The invention discloses a composition, a producing method of the composition and a method for using the composition to treat cancer, which can improve the conventional cancer therapy technologies, such as the side effects of chemotherapy and radiation therapy, and avoid the complex limitations as providing thermotherapy to the entire body. The preparation of the composition of the invention is easy, so that the cost for cancer treatment can be decreased. In addition, the present invention has extremely high cost effectiveness so as to prevent the drawbacks of the conventional physical therapy, which needs a lot of manpower and time, and has poor effect.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A composition used for thermotherapy, comprising:
a carrier structure, comprising:
a lipid layer,
a positive charged polymer dispersed on the lipid layer by non-covalent bonding, and
a surface active polymer dispersed on the lipid layer by non-covalent bonding; and
a plurality of metal particles encapsulated in the carrier structure.

2. The composition of claim 1, wherein the carrier structure comprises liposome.

3. The composition of claim 1, wherein the carrier structure comprises temperature-sensitive liposome.

4. The composition of claim 1, wherein the ratio of the lipid layer, the positive charged polymer and the surface active polymer is between 3:1:1 and 60:1:1.

5. The composition of claim 1, wherein the lipid layer is a neutral lipid layer comprising DLPC, DOPC, DMPC, DPPC, DSPC, DOPC, DMPE, DPPE, DOPE, DMPA, DPPA, DOPA, DMPG, DPPG, DOPG, DMPS, DPPS, or DOPS.

6. The composition of claim 1, wherein the positive charged polymer comprises polyamine, polyethylenimine (PEI), polyvinylpyrrolidone, or polyacetic acid.

7. The composition of claim 1, wherein the surface active polymer comprises crosslinked type polyacrylate salt, saponin, or Polyethylene glycol (PEG).

8. The composition of claim 1, wherein the carrier structure further comprises a targeted subject dispersed on the lipid layer by non-covalent bonding.

9. The composition of claim 8, wherein the targeted subject comprises antibody, peptide, nucleic acid, or cytokine.

10. The composition of claim 1, which is applied in magnetic resonance thermotherapy, wherein the metal particles are magnetic metal particles.

11. The composition of claim 1, wherein the metal particles are made of iron, gold, or copper.

12. The composition of claim 1, wherein the metal particles are subjected to a magnetic force or an electromagnetic wave to generate heat.

13. A producing method of a composition used for thermotherapy, comprising the steps of:
forming a lipid layer;
adding a solution containing a positive charged polymer and a solution containing a surface active polymer to contact with the lipid layer;
shaking the lipid layer with the solutions to form a carrier structure; and
encapsulating a plurality of metal particles in the carrier structure.

14. The method of claim 13, further comprising a step of:
adding a solution containing a targeted subject to contact with the carrier structure.

15. The method of claim 13, wherein the lipid layer comprises temperature-sensitive liposome.

16. The method of claim 13, wherein the composition is applied in magnetic resonance thermotherapy, and the metal particles are magnetic metal particles.

17. The method of claim 13, wherein the metal particles are subjected to a magnetic force or an electromagnetic wave to generate heat.

18. A method for using a composition used for thermotherapy to treat cancer, wherein the composition comprises a carrier structure comprising a lipid layer, a positive charged polymer dispersed on the lipid layer by non-covalent bonding and a surface active polymer dispersed on the lipid layer by non-covalent bonding, and a plurality of metal particles encapsulated in the carrier structure.

19. The method of claim 18, wherein the carrier structure comprises temperature-sensitive liposome.

20. The method of claim 18, wherein the ratio of the lipid layer, the positive charged polymer and the surface active polymer is between 3:1:1 and 60:1:1.

21. The method of claim 18, wherein the composition is applied in magnetic resonance thermotherapy, wherein the metal particles are magnetic metal particles.

22. The method of claim 18, wherein the metal particles are subjected to a magnetic force or an electromagnetic wave to generate heat.
